(19) United States
(12) Patent Application Publication
(10) Pub. No.: US 2024/0115673 A1
(43) Pub. Date: Apr. 11, 2024

(54) METHOD AND COMPOSITION FOR THE TREATMENT OF CANCER

(71) Applicant: AntiCancer Inc., San Diego, CA (US)

(72) Inventors: Yutaro Kubota, San Diego, CA (US); Qinghong Han, San Diego, CA (US); Robert Hoffman, San Diego, CA (US)

(21) Appl. No.: 18/482,386

(22) Filed: Oct. 6, 2023

Related U.S. Application Data
(60) Provisional application No. 63/413,880, filed on Oct. 6, 2022.

(51) Int. Cl. A61K 38/51; A61P 35/00; C12N 15/70

(52) U.S. Cl. CPC A61K 38/51; A61P 35/00; C12N 15/70; C12R 2001/19; C12Y 404/01011

(57) ABSTRACT

A composition and method for treating cancer in a subject in need thereof. The composition includes recombinant-methioninase-producing bacterial strains that upon oral administration can release recombinant-methioninase in the gut of the subject. The bacterial strains include Escherichia coli bacteria comprising the cloned rMETase gene from Pseudomonas putida using plasmid pATG3131, such as Escherichia coli JM109 strain. The bacteria can be incorporated in the microbiome of the subject and release the recombinant-methioninase.

### CROSS-REFERENCE TO RELATED APPLICATIONS

This application claims priority from a U.S. provisional patent application Ser. No. 63/413,880, filed on Oct. 6, 2022, which is incorporated herein by reference in its entirety.

### FIELD OF INVENTION

The present invention relates to a composition and method for treating cancer, and more particularly, the present invention relates to an oral composition containing recombinant-methioninase-producing Escherichia coli.

### BACKGROUND

It has been established from several studies that Cancer cells are generally addicted to methionine due to excess transmethylation reactions occurring in cancer cells. Therefore, cancer cells require a greater amount of methionine than normal cells. Cancer cells cannot survive under conditions of methionine restriction. This specific feature of methionine addiction is a fundamental and general hallmark of cancer and is termed the Hoffman effect. Under the condition of methionine restriction, cancer cells are arrested in the late S/G2 phase of the cell cycle. Methionine restriction is effective against cancer in vitro and in vivo. Methionine restriction of cancer cells sensitizes them to cytotoxic chemotherapy, possibly due to the late S/G2 cell cycle arrest. Also, a study showed that methionine restriction disrupts the flux of one-carbon metabolism and affects vulnerabilities involving redox and nucleotide metabolism in the patient-derived xenograft model and human studies. Dietary methionine restriction combined with cytotoxic agents showed efficacy over the cytotoxic agent alone in clinical trials of melanoma, glioma, colorectal cancer, and gastric cancer. However, all protein sources contain methionine, and it is difficult for patients to restrict methionine strictly by controlling diet alone. Studies have shown that recombinant-methioninase (rMETase) is effective which acts by breaking down methionine in the body. rMETase is produced by fermentation of recombinant Escherichia coli transformed with the methioninase gene from Pseudomonas putida (P. putida). Although proven to be effective, the administration of rMETase by intravenous or intra peritoneal injection has been tedious and uncomfortable and can lead to anaphylaxis.

A need is therefore appreciated for a composition and method that overcome the aforesaid drawbacks in the administration of recombinant-methioninase (rMETase).

### SUMMARY OF THE INVENTION

The following presents a simplified summary of one or more embodiments of the present invention in order to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an oral composition and method for administering the composition.

It is another object of the present invention that frequent dosing can be avoided.

Still, another object of the present invention is that patient compliance can be improved.

In one aspect, disclosed is a method for treating cancer in a subject in need thereof, the method comprising administering, orally, to a subject, a composition comprising recombinant-methioninase-producing bacterial strains in an effective amount to release recombinant-methioninase in a gut of the subject. The bacterial strains comprise Escherichia coli bacteria containing the cloned rMETase gene from Pseudomonas putida using plasmid pATG3131. The Escherichia coli bacteria comprise the Escherichia coli JM109 strain. The recombinant-methioninase enzyme is released at least in an amount sufficient for treating or controlling growth of cancer in the subject. The cancer is colon cancer, breast cancer, or any other cancer. The effective amount of the composition is sufficient to allow gut colonization.

In one aspect, disclosed is a method of generating recombinant-methioninase enzyme in the gut of a subject, the method comprises administering, orally, to the subject, a composition comprising recombinant-methioninase-producing bacterial strains in an effective amount to release recombinant-methioninase enzyme in the gut of the subject. The bacterial strains comprise Escherichia coli bacteria containing the cloned rMETase gene from Pseudomonas putida using plasmid pATG3131. The Escherichia coli bacteria comprise the Escherichia coli JM109 strain. The recombinant-methioninase enzyme is released at least in an amount sufficient for treating or controlling growth of a cancer in the subject. The cancer is colon cancer, breast cancer, or any other cancer. The effective amount of the composition is sufficient to allow gut colonization.

In one aspect, disclosed is an oral composition for treating cancer in a patient in need thereof, the composition comprises recombinant-methioninase-producing bacterial strains in an effective amount to release recombinant-methioninase in a gut of the subject. The bacterial strains comprise Escherichia coli bacteria containing the cloned rMETase gene from Pseudomonas putida using plasmid pATG3131. The Escherichia coli bacteria comprise the Escherichia coli JM109 strain. The recombinant-methioninase enzyme is released at least in an amount sufficient for treating or controlling growth of a cancer in the subject. The cancer is colon cancer, breast cancer, or any other cancer. The effective amount of the composition is sufficient to allow gut colonization.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a graph showing the tolerability of C57BL/6 mice to recombinant-methioninase (rMETase)-producing Escherichia coli JM109 (Escherichia coli JM109-rMETase) as shown by body weight.

FIGS. 2A-2C show the efficacy of recombinant-methioninase (rMETase)-producing Escherichia coli JM109 (Escherichia coli JM109-rMETase) on the syngeneic MC38 colon-cancer in the C57BL/6 mouse model, FIG. 2A shows a tumor growth curve, FIG. 2B shows the tumor size on day 15, and FIG. 2C shows the body weight change.

FIG. 3 shows the photographs of tumors excised from mice treated with phosphate-buffered saline (control), the photographs of tumors excised from mice treated with the control Escherichia coli JM109 competent cells, and the photographs of tumors excised from mice treated with recombinant-methioninase-expressing Escherichia coli JM109-rMETase cells.

FIG. 4 is a graph showing the efficacy of recombinant-methioninase (rMETase)-producing Escherichia coli JM109 (Escherichia coli JM109-rMETase) against the 4T1 mouse triple-negative breast cancer in an athymic nu/nu nude-mouse orthotopic model on day 15.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention since the scope of the invention will be best defined by the allowed claims of any resulting patent.

Disclosed is a composition including rMETase-producing Escherichia coli JM109 (Escherichia coli JM109-rMETase), referred to herein as Escherichia coli JM109. The disclosed composition can be administered orally to a patient or subject in need thereof. The disclosed composition can be administered orally for delivery into the intestine of the subject, wherein Escherichia coli JM109 can release rMETase into the gut. The release of rMETase can degrade the methionine in the body reducing the concentration thereof and thus helping in the treatment of cancer. The disclosed composition can be used for the treatment of various types of cancers by reducing the methionine levels in the body. Specifically, the disclosed composition by releasing the rMETase into the gut can be effective in controlling and treating colon cancers. The rMETase may continuously be released by the administered Escherichia coli JM109 into the body, and the effective levels of rMETase can be maintained in the body. Frequent administration of methioninase enzyme orally or by any other route can be avoided, thus improving patient compliance and comfort, while improving the effectiveness of treatment. It is understood that the disclosed composition may include any other bacteria that is safe for administration to humans and can produce methioninase enzyme in the gut in effective amounts for the treatment of cancer. The use of Escherichia coli JM109 is only provided as an example and any such organism can be incorporated into the disclosed composition without departing from the scope of the present invention. A composition and method are disclosed for the treatment of cancer in a patient by administering methioninase-producing bacteria through the oral route. The administered Escherichia coli JM109 or similar bacteria may get incorporated into the microbiome of the subject, thus further reducing the need for frequent administration of the composition.

The disclosed composition may include the Escherichia coli JM109 in safe amounts, or the amounts considered safe for administration to humans. Such an amount can be determined clinically and can be optimized through clinical studies. For example, a tolerability study demonstrated that up to 2×10¹⁰ CFU/day of Escherichia coli JM109-rMETase is tolerable. In a separate study, it was found that Escherichia coli JM109-rMETase (10¹¹ CFU/ml), after exposure to Isopropyl β-d-1-thiogalactopyranoside (IPTG), had rMETase activity of 39.7 U/ml, which is about one-tenth of the rMETase usually used for mice (500 U/ml) for oral administration.

### EXPERIMENTATION

Culture of Escherichia coli JM109-rMETase:

Escherichia coli JM109 was used as the host strain for the expression of rMETase. The rMETase gene from P. putida was cloned into Escherichia coli JM109 using plasmid pATG3131, which also includes the tetracycline (TC)-resistance gene. The resulting Escherichia coli JM109-rMETase was pre-cultured in 5 ml of Luria-Bertani (LB) liquid medium with TC (32 μg/ml) for 8 hours at 37° C. Preculture broth was transferred to a 400 ml culture medium with TC (32 μg/ml) and cultivated overnight. After culture, isopropyl-β-D-thiogalactopyranoside (IPTG) was added at a final concentration of 0.3 mM for 4 hours at 28° C. to induce expression of rMETase. The optical density at 600 nM was used to determine the amount of live Escherichia coli JM109-rMETase in the medium. After manually counting colonies, it was determined that an O.D. 600 of 0.7 comprises approximately 1.0×10⁹ colony-forming units (CFU)/ml of Escherichia coli JM109-rMETase. Escherichia coli JM109-rMETase was harvested and diluted with phosphate-buffered saline (PBS), and 20% glycerin was added for storage at −80° C. until administration to mice.

Escherichia coli JM109 competent cells were prepared the same as Escherichia coli JM109-rMETase as a control but without TC in the LB medium, and IPTG was not added to the culture.

Escherichia coli JM109-rMETase Tolerability Study in C57BL/6 Mice:

Firstly, a preliminary study was conducted to evaluate the tolerability of the C57BL/6 mouse to Escherichia coli JM109-rMETase. Three different doses of Escherichia coli JM109-rMETase (10⁶, 10⁸, and 10¹⁰ CFU/100 μl) were prepared and fed by oral gavage to healthy C57BL/6 mice in the morning and evening, for 1 week, and the weight of each mouse was assessed.

rMETase Activity Assay

rMETase activity was determined from α-ketobutyrate produced from L-methionine, as previously reported. rMETase activity produced by different amounts of Escherichia coli JM109-rMETase was examined. Assuming an intestinal environment, 20 mM deoxycholic acid was added to Escherichia coli JM109-rMETase treated with IPTG, as described above, in order to release the rMETase from the bacteria. After the addition of 0.5 mM pyridoxal 5′-phosphate and 0.5 mg/ml dithiothreitol for 3 hours, rMETase activity was evaluated.

rMETase activity in Escherichia coli, cultured with IPTG, from mouse stool was also Examined. The bacterial density was adjusted, and bacteria were sonicated for 30 seconds after adding pyridoxal 5′-phosphate and dithiothreitol, and then rMTEase activity was evaluated.

### Animals and Diets

C57BL/6 mice (AntiCancer Inc., San Diego, CA, USA), aged 4-6 weeks old, were used in the study. The mice were housed in a barrier facility with a HEPA-filtered rack under typical light/dark cycles of 12 hours. An autoclaved laboratory rodent diet was fed to mice from 9 a.m. to 5 p.m. during this study. Approval was received from the AntiCancer Institutional Animal Care and Use Committee's ethical committee under National Institutes of Health Guide Assurance Number 3873-1. All experiments were performed in accordance with the Animal Research: Reporting of In Vivo Experiments (ARRIVE) 2.0 criteria.

### Cell Culture:

The MC38 mouse colon cancer cell line was cultured in high-glucose Dulbecco's modified Eagle's medium (DMEM) supplemented with 10% fetal bovine serum and 100 IU/ml penicillin/streptomycin at 37° C. in a humidified environment containing 5% carbon dioxide.

### Establishment of Subcutaneous Tumors:

Eighteen C57BL/6 male mice were injected subcutaneously with 10⁶ MC38 cells in the right flank. One week after injection, subcutaneous tumors were established.

### Treatment Study Design:

Mice implanted with MC38 subcutaneously were randomized into three groups of six mice each: Group 1: Untreated control given 100 μl PBS orally twice daily (9 a.m. and 5 p.m.) for 14 days; Group 2: Escherichia coli JM109 competent cells (10¹⁰/100 μl) orally by gavage as a control twice daily (9 a.m. and 5 p.m.) for 14 days: Group 3: Escherichia coli JM109-rMETase cells (10¹⁰/100 μl) orally by gavage twice daily (9 a.m. and 5 p.m.) for 14 days. TC (0.5 g/l) was added to the mouse's drinking water to prevent plasmid shedding only in group 3 (29). Tumor volume and body weight were measured every 2 days during the treatment. Tumor volume (mm³) was calculated with the following formula: Tumor volume (mm³)=length (mm)×width (mm)×width (mm)×½.

### Stool and Tissue Collection:

Mouse stool was collected before treatment (day 0) and on the evening of day 14. The mice were sacrificed on day 15, and the tumor was removed.

### Mouse Stool Culture:

Stool culture was prepared to examine whether Escherichia coli JM109-rMETase was incorporated into the mouse microbiome or not. The stool was diluted 1:10 by weight into PBS and then homogenized by mechanical disruption. Large debris was pelleted with short centrifugation at 200×g. Then 100 μl of supernatant was plated onto LB agar with 32 μg/ml TC and incubated overnight at 37° C.

Screening for Escherichia coli JM109-rMETase in Mouse Stool Culture:

Gram staining of colonies formed from stool culture was performed. The Gram-negative bacterial colonies were plated on a modified M9 medium with TC (32 μg/ml) and IPTG (0.3 mM) to determine whether these bacteria produced rMETase or not. The modified M9 medium contained 6 g/l disodium hydrogen phosphate, 3 g/l potassium dihydrogen phosphate, 0.5 g/l sodium chloride, 5 g/l L-methionine, 0.24 g/l magnesium sulfate, 0.011 g/l calcium chloride, 2 g/l glucose, 32 μg/ml TC, and 0.3 mM IPTG. Before pouring the plates, phenol red was added to the medium as an indicator for methioninase at a final concentration of 0.007% (w/v) at a pH of 7.0 (59,60). It was determined whether colonies produced rMETase based on the pink color of the colonies because ammonium, produced from methionine by rMETase, makes the colony pink due to phenol red in the modified M9 medium.

### Statistics:

All statistical analyses were performed with GraphPad Prism 9.4.0 (GraphPad Software, Inc., San Diego). Tukey-Kramer was performed for the parametric test of comparison between groups. All data are displayed as the mean±standard deviation. p-Values≤0.05 were regarded as significant.

### Results:

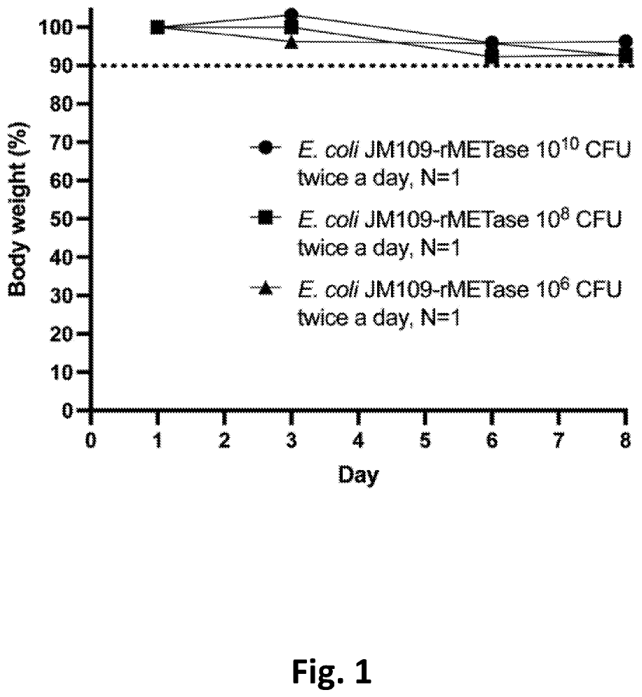

Tolerability of Escherichia coli JM109-rMETase in C57BL/6 mice. Different amounts of Escherichia coli JM109-rMETase administered orally in C57BL/6 mice for 1 week allowed the body weight of mice to be maintained at all amounts tested, indicating Escherichia coli JM109-rMETase was therefore not toxic (FIG. 1).

rMETase Activity at Different Escherichia coli JM109-rMETase Cell Densities:

The rMETase activity of 10¹¹ CFU/ml Escherichia coli JM109-rMETase was 39.7 U/ml compared to 7.9 U/ml of 10¹⁰ CFU/ml Escherichia coli JM109-rMETase. Based on these results, the dose of Escherichia coli JM109-rMETase to be used in subsequent experiments was determined to be 10¹⁰ CFU/100 μl every morning and evening for 2 weeks.

Antitumor Efficacy of Oral Escherichia coli JM109-rMETase:

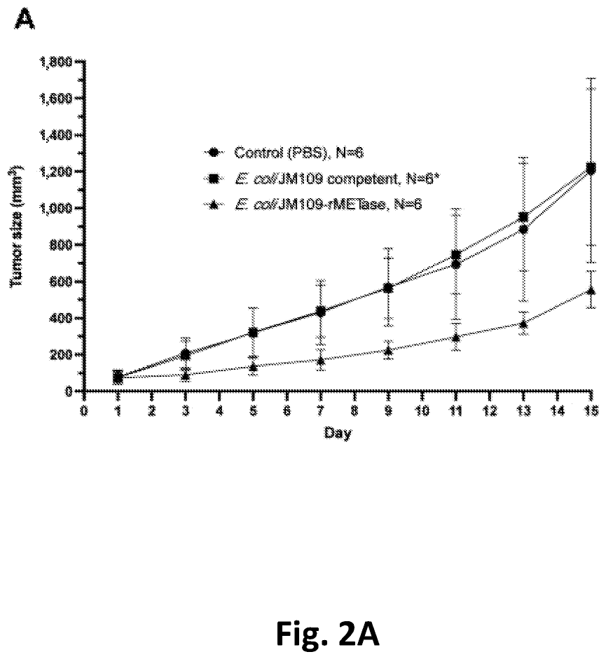

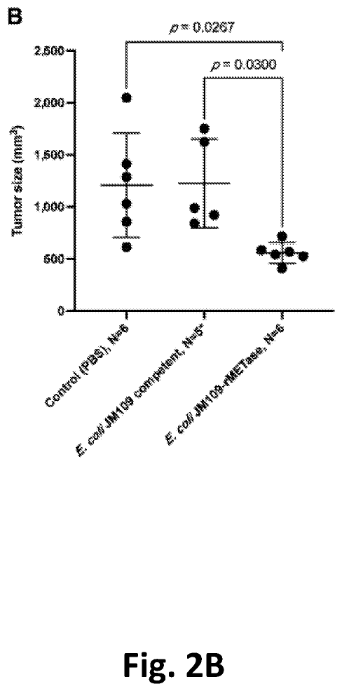

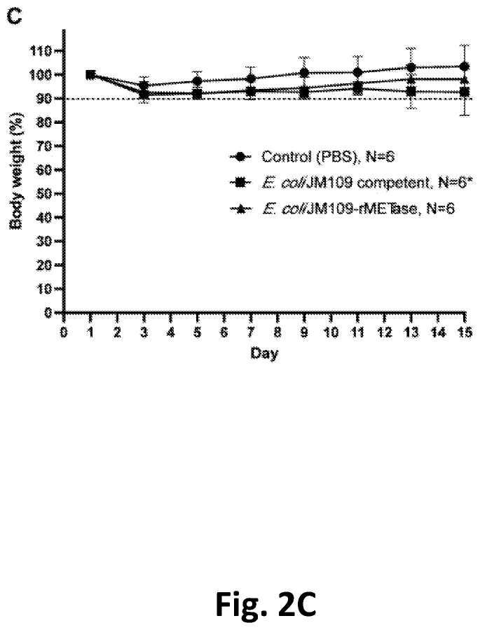

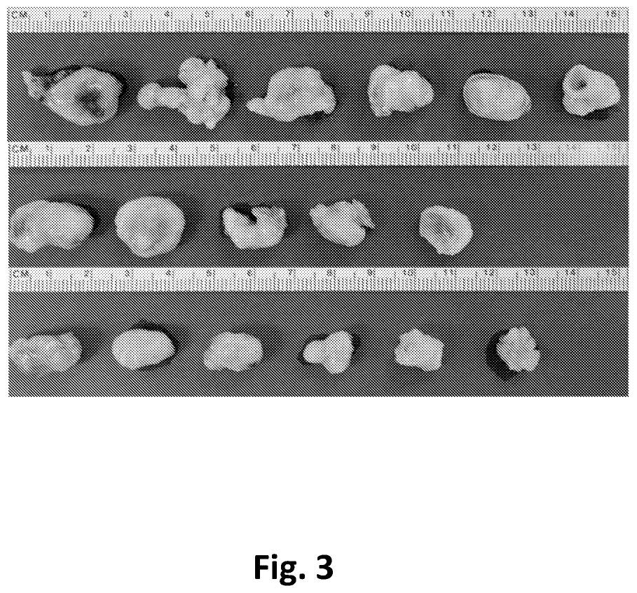

Escherichia coli JM109-rMETase significantly inhibited MC-38 tumor growth in C57BL6 mice compared to the untreated control or non-rMETase-producing Escherichia coli JM109 competent cells (control vs. Escherichia coli JM109-rMETase: p=0.0267; Escherichia coli JM109 competent cells vs. Escherichia coli JM109-rMETase: p=0.03) (FIGS. 2A and 2B, and FIG. 3).

### Body Weight Changes:

FIG. 2C shows no significant change in mouse body weight for each group, confirming the tolerability of 10¹⁰ CFU/100 μl Escherichia coli.

Stool culture to determine the presence of Escherichia coli JM109-rMETase. The culture of stool bacteria on modified M9 agar resulted in pink colonies of Gram-negative bacilli, consistent with the presence of Escherichia coli JM109-rMETase. Furthermore, after the isolation of these colonies, they were grown to 10¹¹ CFU/ml and examined for rMETase activity. All the pink colonies had rMETase activity (data not shown).

The results indicate that 2×10¹⁰ CFU/day of Escherichia coli JM109-rMETase was found to inhibit MC-38 tumor growth in C57BL6 mice. In the study, Escherichia coli JM109-rMETase was found in mouse stool after 2 weeks of oral administration of Escherichia coli JM109-rMETase, indicating it was incorporated into the microbiome.

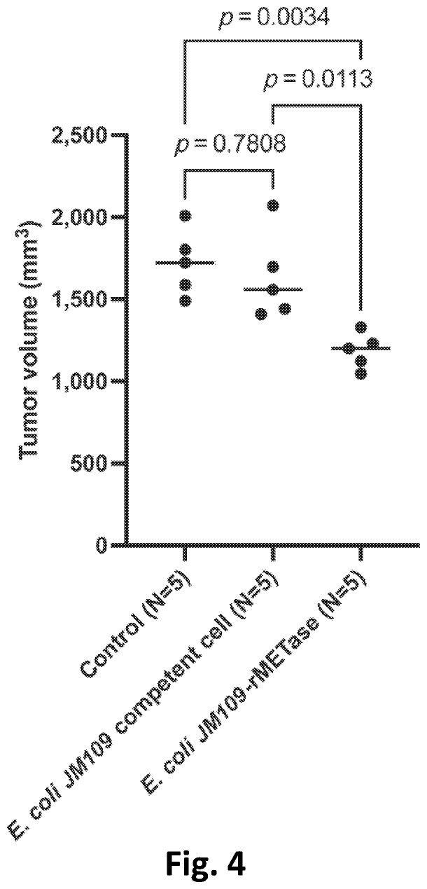

Antitumor efficacy of oral Escherichia coli JM109-rMETase on the growth of 4T1 tumors in athymic nu/nu nude mice was also evaluated. The oral Escherichia coli JM109-rMETase significantly suppressed the growth of triple-negative mouse breast cancer cells (4T1) compared to the PBS-treated control or Escherichia coli JM109 competent cells that do not produce rMETase (p=0.0034 for the PBS control vs. Escherichia coli JM109-rMETase; p=0.0113 for Escherichia coli JM109 competent-cells control vs. Escherichia coli JM109-rMETase) (FIG. 4). Also, the Escherichia coli JM109-rMETase was found to be well tolerated at 10¹⁰ in nude mice. On day 15 of the study, the mean methionine levels were reduced by approximately 30 percent in mice treated with 10¹⁰ Escherichia coli JM109-rMETase.

Thus, Escherichia coli JM109-rMETase lowered blood methionine levels and inhibited triple-negative breast cancer (TNBC) growth in an orthotopic cell-line mouse model, suggesting future clinical potential against a highly recalcitrant cancer. Bacterial therapy using Escherichia coli JM109-rMETase showed growth-inhibitory efficacy against mouse TNBC 4T1, in nude mice.

The disclosed composition and method may help manipulate the gut microbiome to reduce methionine levels in the body, which may help treat recalcitrant cancer. The deoxycholic acid from the liver may also lyse the bacteria, thereby releasing methioninase into the gut. Blood methionine levels were found to be significantly reduced following the therapy using the disclosed composition.

Also disclosed is a formulation containing the methioninase-producing bacteria for administration to a human, preferably, through an oral route, however, any other suitable route of administration is within the scope of the present invention. The optimal amounts of methioninase-producing bacteria can be determined clinically for administration based on various factors, such as the age of the subject. The formulation may also include other ingredients that may help to increase the shelf life of the formulation. Examples of such ingredients chiefly include protectants and additives used to improve the survival rate of the bacteria during manufacture and storage of the formulation. Examples of ingredients include saccharides, skimmed milk, whey proteins, inulin, trehalose, oligosaccharides, and polymers. Other additives, such as antioxidants and taste enhancers, may be added to the compositions. For example, an antioxidant, such as d-alpha-tocopherol, may be added to the formulation. Taste enhancers, such as citric acid, and other natural and artificial flavors may also be added to the formulation. The formulation may also include IPTG or the IPTG can be administered separately, for example with drinking water.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for treating cancer in a subject in need thereof, the method comprising:
administering, orally, to a subject, a composition comprising recombinant methioninase producing bacterial strains in an effective amount to release recombinant methioninase in a gut of the subject.

2. The method of claim 1, wherein the bacterial strains comprise E. coli bacteria having cloned rMETase gene from Pseudomonas putida using plasmid pATG3131.

3. The method of claim 2, wherein the E. coli bacteria comprises E. coli JM109 strain.

4. The method of claim 1, wherein the recombinant methioninase is released at least in an amount sufficient for treating or controlling growth of a cancer in the subject.

5. The method of claim 4, wherein the cancer is colon cancer.

6. The method of claim 4, wherein the effective amount of the composition is sufficient to allow gut colonization.

7. A method of generating recombinant methioninase enzyme in a gut of a subject, the method comprises:
administering, orally, to the subject, a composition comprising recombinant methioninase producing bacterial strains in an effective amount to release recombinant methioninase enzyme in the gut of the subject.

8. The method of claim 7, wherein the bacterial strains comprise E. coli bacteria having cloned rMETase gene from Pseudomonas putida using plasmid pATG3131.

9. The method of claim 8, wherein the E. coli bacteria comprises E. coli JM109 strain.

10. The method of claim 7, wherein the recombinant methioninase enzyme is released at least in an amount sufficient for treating or controlling growth of a cancer in the subject.

11. The method of claim 7, wherein the effective amount of the composition is sufficient to allow gut colonization.

12. An oral composition for treating cancer in a subject in need thereof, the composition comprises:
recombinant methioninase producing bacterial strains in an effective amount to release recombinant methioninase in a gut of the subject.

13. The oral composition of claim 12, wherein the bacterial strains comprise E. coli bacteria having cloned rMETase gene from Pseudomonas putida using plasmid pATG3131.

14. The oral composition of claim 13, wherein the E. coli bacteria comprises E. coli JM109 strain.

15. The oral composition of claim 12, wherein the recombinant methioninase is released at least in an amount sufficient for treating or controlling growth of a cancer in the subject.

16. The oral composition of claim 15, wherein the cancer is colon cancer.

17. The oral composition of claim 1, wherein the effective amount of the composition is sufficient to allow gut colonization.
